(19) United States
(12) Patent Application Publication
(10) Pub. No.: US 2020/0352723 A1
(43) Pub. Date: Nov. 12, 2020

(54) ELECTROMAGNETIC SPINAL CAGE

(71) Applicant: A.I. Spine LLC, Seattle, WA (US)

(72) Inventors: Omar F. Jimenez, Seattle, WA (US); Yefim I. Safris, Golden Valley, MN (US); John McCarty, New Paltz, NY (US)

(21) Appl. No.: 16/871,812

(22) Filed: May 11, 2020

Related U.S. Application Data
(60) Provisional application No. 62/846,139, filed on May 10, 2019.

(51) Int. Cl. A61F 2/28; A61F 2/44; A61F 2/46

(52) U.S. Cl. CPC A61F 2/28; A61F 2/447; A61F 2002/2821; A61F 2002/4683; A61F 2/4611

(57) ABSTRACT

Disclosed herein are electromagnetic enhanced spinal implants inserted into the disc space via a minimally invasive surgical approach. The spinal implants can include one or more internal coils that generate a magnetic field to enhance bone growth. The device can be powered by an external transmitter. The transmitter will provide a minimum voltage and power output that will allow stimulation of the internal coil.

### RELATED APPLICATION

The present application claims the benefit of U.S. Provisional Application No. 62/846,139 filed May 10, 2019, which is hereby incorporated herein in its entirety by reference.

### TECHNICAL FIELD

The present disclosure relates to the fusion of vertebral bodies.

### BACKGROUND

The use of electromagnetic energy (EM) for enhancing the fusion of long bones has been well established in the literature. Studies have demonstrated positive effects of electromagnetic fields. The use of implantable bone stimulating devices and external wearable magnetic field generating devices for the purpose of improving and enhancing spinal fusions have been extensively studied and guidelines for use have been developed. Overall, this technology is supported and encouraged only in patients at high risk of spine fusion pseudarthrosis. It is theorized that EM mimics the mechanical stress on bone by creating pressure gradients at the molecular and DNA level resulting in bone growth and repair.

There are three types of electrical magnetic stimulation for bone fusion currently used: Direct Current Stimulation (DCS), Pulse Electromagnetic Field Stimulation (PEFMS) and Capacitive Coupled Electrical Stimulation (CCES). The only surgically implanted form of electromagnetic energy stimulation for spinal fusions has been DCS. In this procedure cathodes are implanted in the posterolateral exposed transverse processes along the vertebrae with the anode in nearby soft tissue and the battery placed either in the subcutaneous or subfascial plane. Bone formation has been reported to occur at the cathodes at currents of 5-100 uA.

PEFMS and CCES are non-invasive and the electrodes are placed externally on opposite sides of the fracture or fusion area. In CCES, applied potentials are in the range of 1-10V at frequencies of 20-200 KHZ. The resulting magnetic fields in the tissues are in the range of 1-100 mV/cm. In PEMF, a single or double coil is driven by an external field generator producing the necessary current. The configuration of the applied magnetic fields has varied amplitudes, including frequency-single or pulse burst (a serious of pulses with frequencies of 1 to 100 burst/second), and waveforms. Varying configurations have produced magnetic fields of 0.1-20 Gauss (G), which have produced voltage gradients of 1-100 mV/cm.

The Clinical Spine Literature has reported that EM stimulation has proven statistically significant outcomes in bone fusion and Clinical Outcome. The use of Pulse Electromagnetic Field Stimulation and Capacitive Coupled Electrical stimulation has been well documented in the literature as a non-invasive adjunct to spinal fusion. To date DCS, PEFMS and CCES have not been used for direct surgical implantation in the disc space for the purpose of enhancing fusions in the disc space.

There are several biologic materials spine surgeons also can use to promote fusion in spine surgery. These include local autologous laminectomy bone, iliac crest bone graft, cadaveric demineralized bone matrix, porcelain matrix, and synthetic bone morphogenic protein (BMP) (rh-bmp-2). Amongst all options, rh-bmp-2 is very popular with minimally invasive spine surgeons. BMP is easy to reconstitute, implant and has clinical data demonstrating success both radiographically with successful spinal fusions and clinical improvement pain scores. However, the benefits of BMP are not without risk. Formation of heterotopic bone can result in nerve root compression, bone lysis and negative fusion effects, nerve root irritation and radiculopathy. Also, in patients with a previous history of cancer, there is the potential risk of inciting malignancy, although, the literature has not affirmed this risk. Moreover, BMP is very expensive and a large dose for a multi-level fusion increases the overall cost of surgery. Comparison of alternative fusion enhancers to BMP, in particular, DCS implantation for spine fusions have yielded similar outcomes both in fusion rates and clinical outcomes.

With the improvement of Minimally Invasive Spine Surgical techniques and equipment, transforaminal lumbar interbody fusion, direct lateral and anterior interbody fusion approaches have become critical strategic access tools for minimally invasive spine surgeons. That is, spinal cages are implanted in the disc space via different minimal access surgical corridors, adjacent to vertebrae endplates that have been decorticated for the purpose of bone fusion. Although, posterior techniques addressing the facets and pars are important, the interbody fusion cages help in providing additional surface area for bone fusion, and for correction of spinal deformities. Because in some cases minimal access surgeons are relying only on the interbody fusion, it is imperative that every attempt be made to enhance bone fusion.

### SUMMARY

Disclosed herein are electromagnetic enhanced spinal implants inserted into the disc space via a minimally invasive surgical approach. The spinal implants can include one or more internal coils that generate a magnetic field to enhance bone growth. The device can be powered by an external transmitter. The transmitter will provide a minimum voltage and power output that will allow stimulation of the internal coil.

In an embodiment, an electromagnetic spinal cage configured to be implanted into an intervertebral disc space in a patient's body, includes a device body having an interior chamber therein and having an open bone chamber defined therethrough. A metallic coil can be disposed within the interior chamber and around the bone chamber. A power source can be configured to receive energy from an external transmitter and to supply power to the metallic coil to cause the metallic coil to generate a magnetic field within the bone chamber.

In an embodiment, a system for spinal surgery can include an electromagnetic spinal cage configured to be implanted into an intervertebral disc space in a patient's body and an external power source. The electromagnetic spinal cage can include a device body having an interior chamber therein and having an open bone chamber defined therethrough and a metallic coil disposed within the interior chamber and around the bone chamber. The external power source can be configured to transfer energy to the electromagnetic spinal cage to power the metallic coil to cause the metallic coil to generate a magnetic field within the bone chamber.

### BRIEF DESCRIPTION OF THE DRAWINGS

Subject matter hereof may be more completely understood in consideration of the following detailed description of various embodiments in connection with the accompanying figures, in which:

FIGS. 1A-2E depict an electromagnetic cage according to the disclosure.

FIGS. 3A-5D depict an electromagnetic cage according to the disclosure.

FIGS. 6A-7 depict an electronic magnetic implant according to the disclosure.

FIGS. 8A-8C depict an electromagnetic implant inserted into a bone matrix material within a bone chamber of an intervertebral cage according to the disclosure.

FIGS. 9A-9E depict a delivery system for an electromagnetic implant according to the disclosure.

FIGS. 10A-10H depict an electromagnetic cage according to the disclosure.

FIG. 11 depicts a schematic representation of an electromagnetic cage system according to the disclosure.

FIG. 12 depicts a schematic representation of an electromagnetic cage system according to the disclosure.

FIG. 13 depicts a schematic representation of an electromagnetic cage system according to the disclosure.

FIG. 14 depicts a piezoelectric energy harvesting circuit according to the disclosure.

FIGS. 15A-15B schematically depict a physical implementation of an electromagnetic cage according to the disclosure.

FIGS. 16A-16D depict schematic representations of spinal surgery systems according to the disclosure.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

As noted above, it is imperative that every attempt be made to enhance bone fusion. In this regard, the use of magnetic field generating devices implanted in the disc space whether in a cage or in a bone matrix will add another option to enhance bone fusions particularly when BMP, bone allograft or autograft is not an option. Having this technology that directly enhances fusion in the disc space will further expand bone fusion alternatives. Embodiments disclosed herein relate to an electromagnetic enhanced bone matrix, with a cage option, that is inserted into the disc space via a minimally invasive surgical approach. The Bone Matrix has multiple internal coils that generate a magnetic field. The coils are connected in parallel to piezo transducers, an MCU w/Bluetooth, MosFET, and a Drop out regulator. The device whether an electromagnetic Bone Matrix device or cage will be powered by an external transmitter. The transmitter will provide a minimum voltage and power output that will allow stimulation of the internal controllers and coil subsystem.

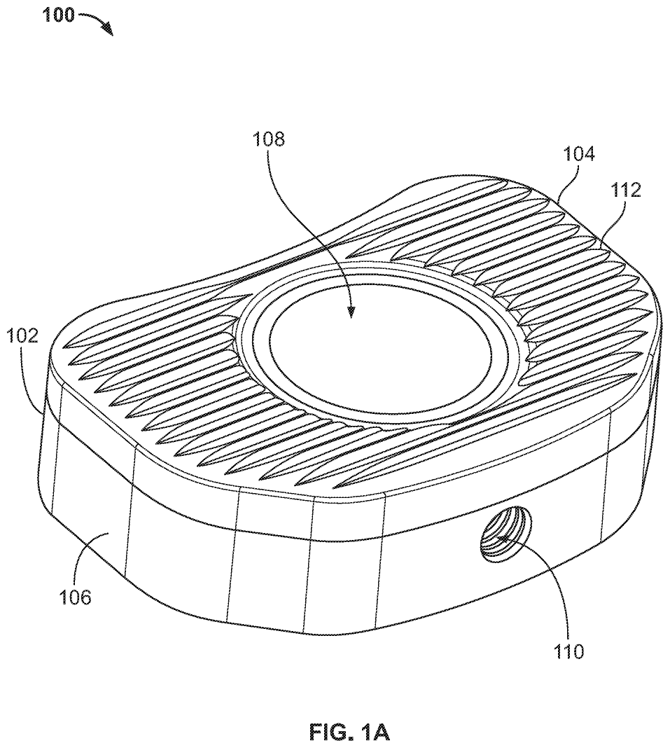

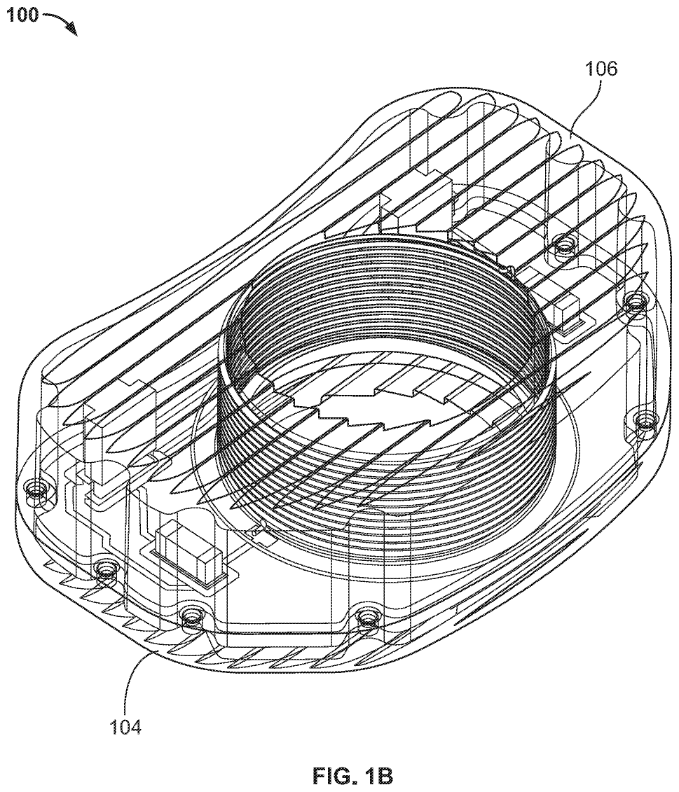

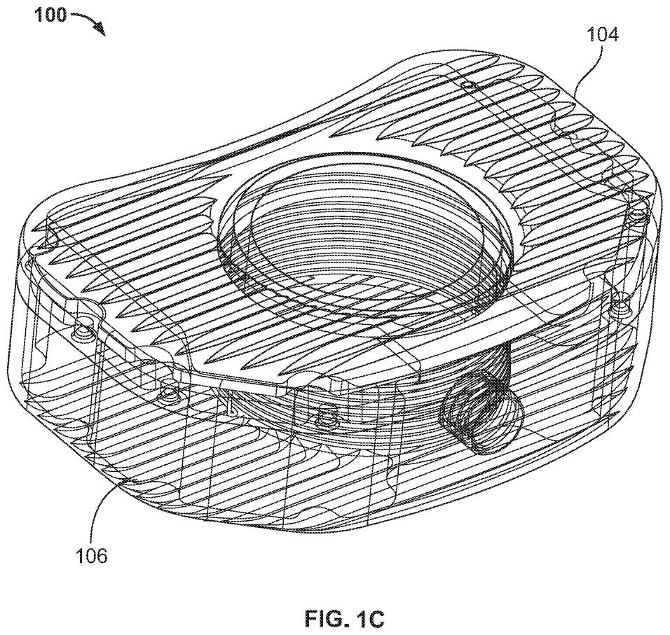

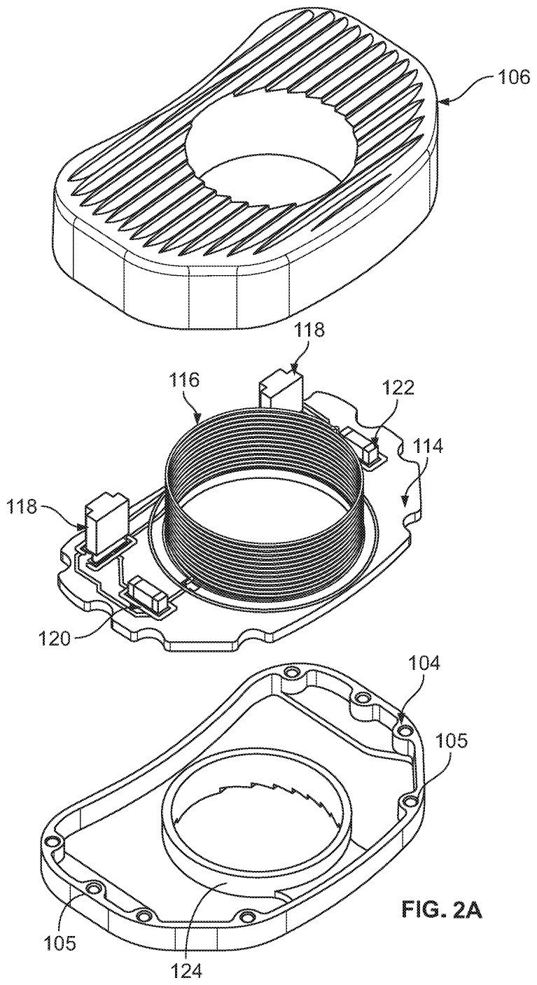

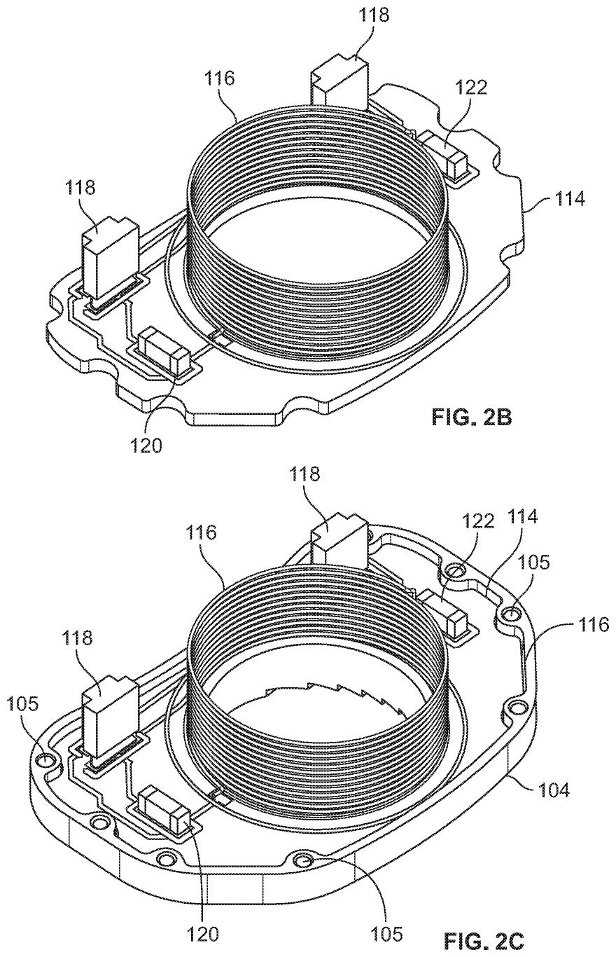

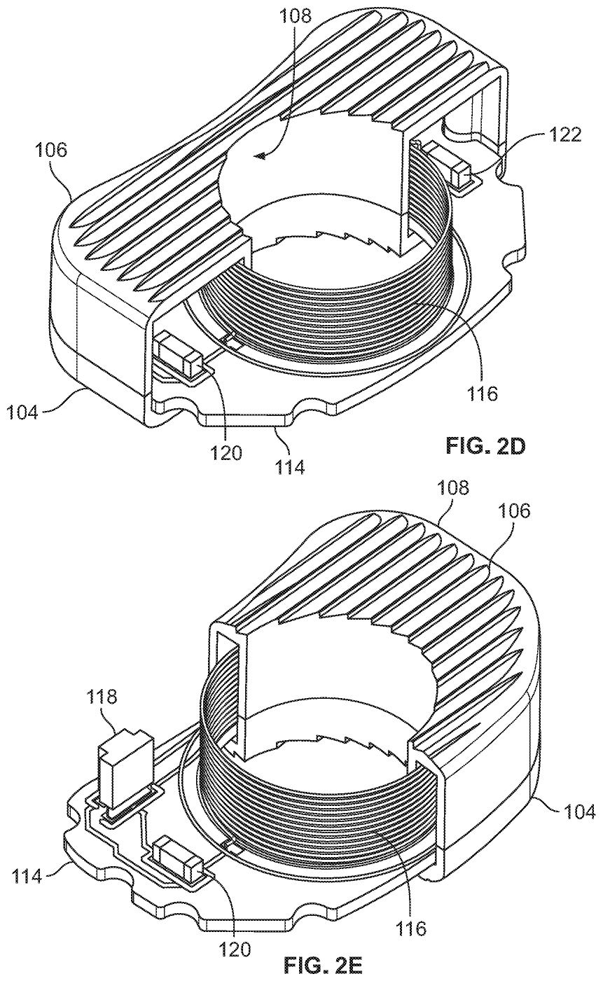

FIGS. 1A-2E depict an electromagnetic (EM) cage 100 configured for implantation into an intervertebral space of a patient to support and promote fusion among adjacent vertebrae according to the disclosure. In embodiments, electromagnetic cage 100 can include a body 102 having a first or upper portion 106 and a second or lower portion 104 (note that FIGS. 1A and 1C depict the implant with the lower portion 104 on top, whereas FIG. 1B depicts the orientation in which the device would be implanted with the upper portion 106 on top). In embodiments, cage 100 is sized and shaped for use as an Anterior Lumbar Interbody Fusion (ALIF) cage. For example, body can have a width of 35 to 40 mm, a depth of 25 to 30 mm and a height of 7 to 12 mm.

A hollow chamber 108 can be formed in body 102 and extend partially or completely through upper portion 106 and/or lower portion 104. In embodiments, the hollow chamber 108 can be configured to receive bone graft material to aid in bone fusion. An inserter recess 110 can also be defined in body 102 to receive a distal end of an insertion tool used to insert the EM cage 100 into the body through the surgical access opening. The inserter recess 100 may be threaded in order to releasably secure the EM cage 100 to the inserter. In embodiments, the upper portion 106 and/or lower portion 104 can have a textured outer bearing surface 112 to provide grip and traction against the bone to prevent accidental movement or withdrawal of the EM cage 100 and/or to promote bone growth into bearing surface 112 to better retain EM cage 100. In embodiments, the upper 106 and lower 104 portions of body 102 can be held together by fasteners, such as screws, inserted through apertures 105 in lower portion 104 (see FIG. 2A and FIG. 2C) and into upper portion 106. In other embodiments upper and lower portions can be held together by other means, such as, for example soldering or glue.

Referring now to FIGS. 2A-2E, EM cage 100 includes electrical components within a hollow chamber defined within body 102 to provide the electromagnetic field that aids in stimulating bone growth within and/or around the bone chamber 108. The components are mounted on a base or breadboard 114 sized and shaped with an outer perimeter that matches an internal perimeter 116 of the lower portion 104 of the EM cage 100. Internal perimeter 116 of lower portion 104 can be raised to prevent movement of breadboard 114 within EM cage 100. In embodiments, the components mounted on the breadboard 114 can include a metallic coil 116 for transmitting the electromagnetic energy, a pair of ultrasonic transducers 118 that transfer power to the coil 116, a power supply 120 that provides power to the transducers 118 and a MCU (microcontroller) chip 122 that can control device operations including in embodiments wireless communications such as by Bluetooth, for example. The coil 116 can be sized to fit around a raised channel perimeter 124 in lower portion 104 and/or upper portion 106 of EM cage 100 and have a generally circular configuration defining an open interior that matches hollow chamber 108. In an embodiment, the coil 116 can have a diameter of approximately 10 mm.

In embodiments, the ultrasonic power transducers 118 can be a piezo-electric energy circuit that will achieve up to 70% efficiency providing an average of 5-10 mw of power within a 1 cm² area. In embodiments, the power consumption of the controller 120 and MCU 122 chips can be configured to minimize power consumption. In addition, the frequency that drives the coils can be kept below 100 khz to minimize energy consumption. In one embodiment, a peak magnetic field of 13 mT is achieved with 100 mW of power provided to a coil having N=100 turns with DC stimulation.

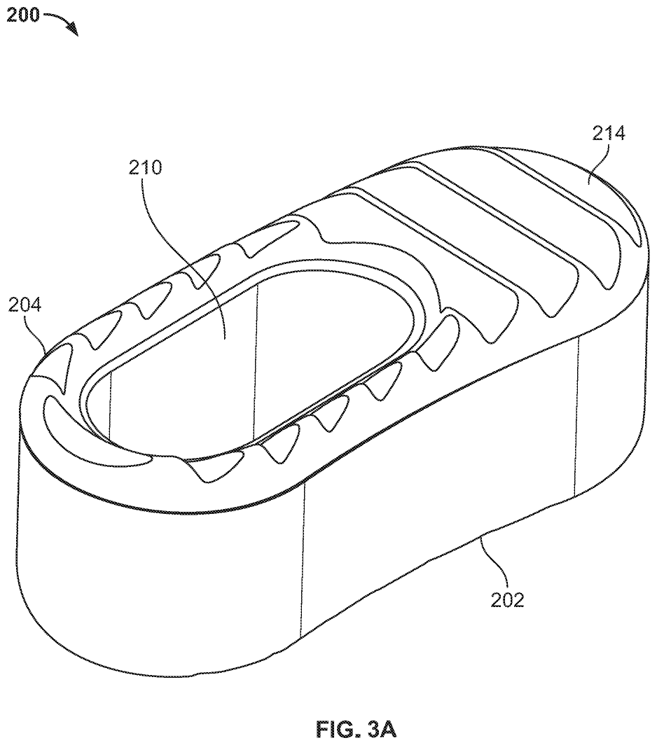

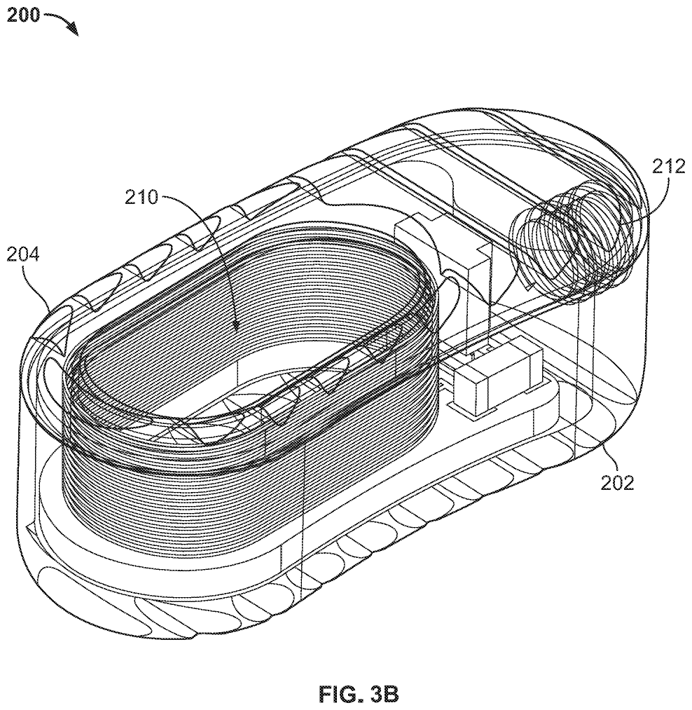

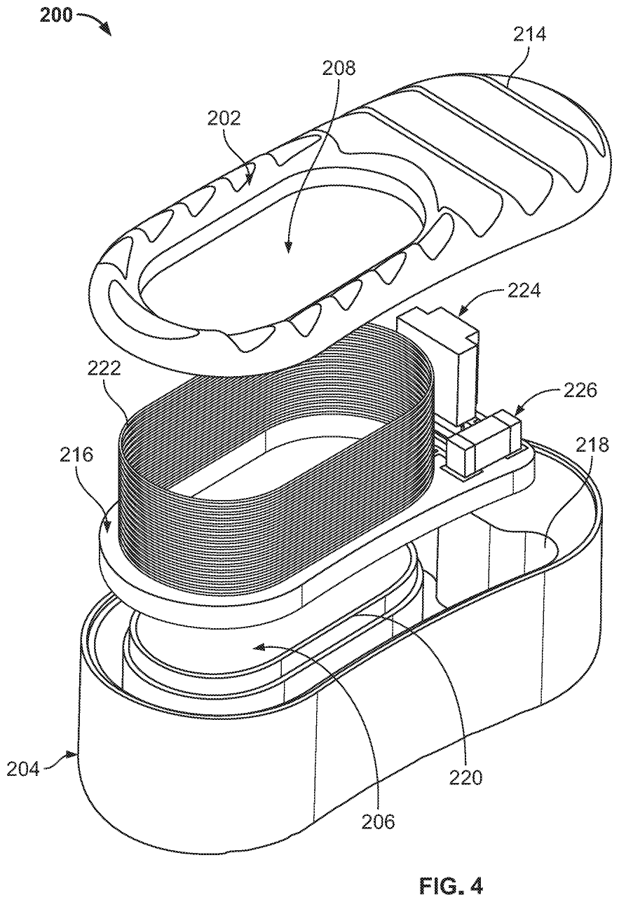

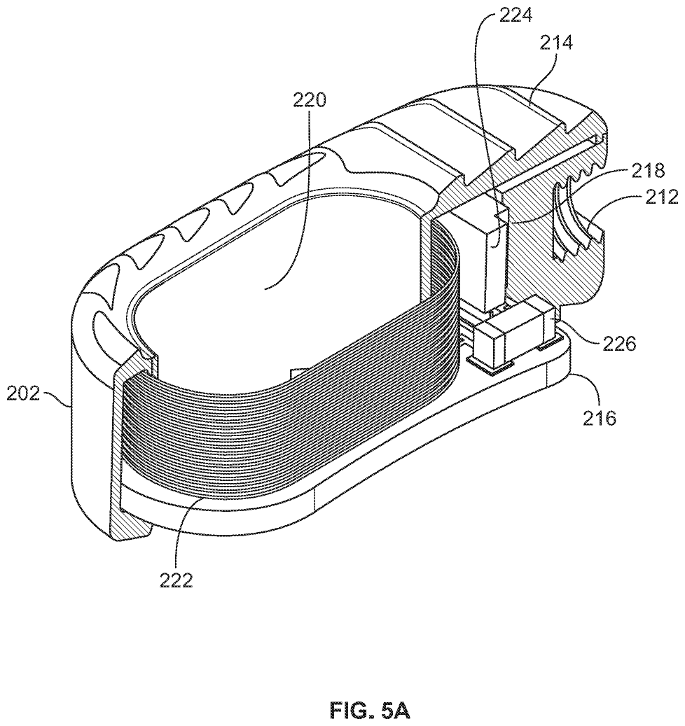

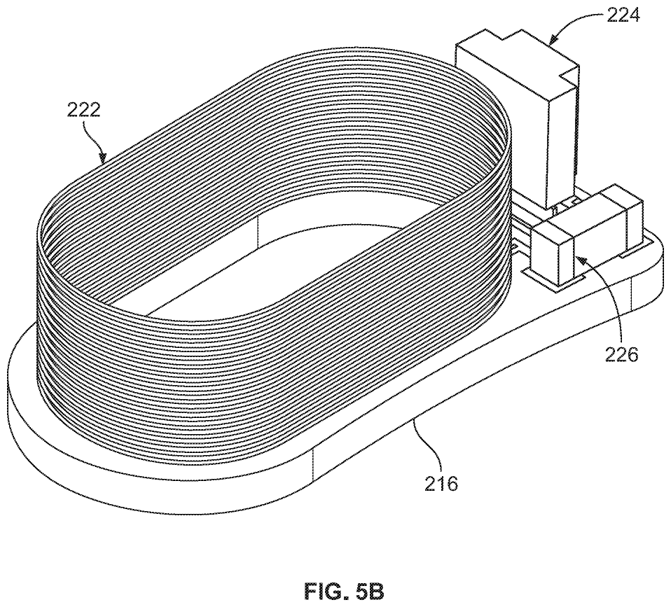

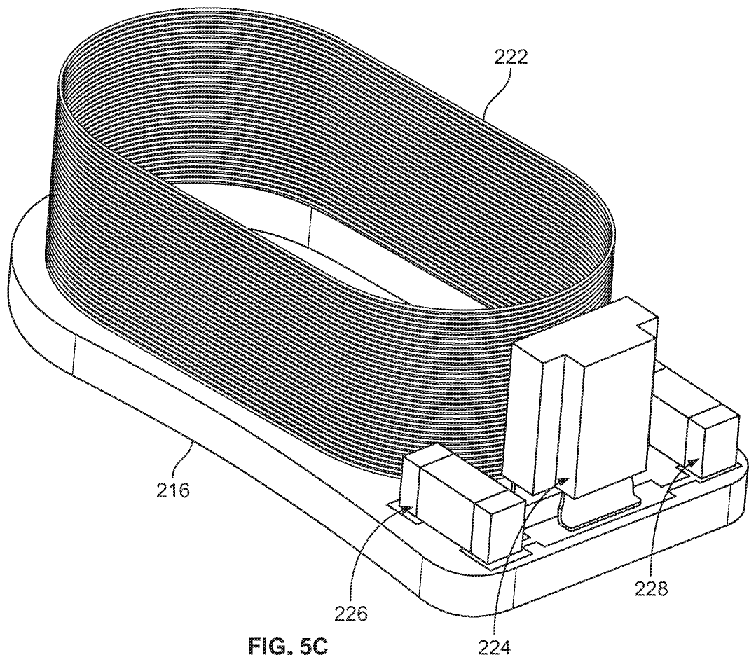

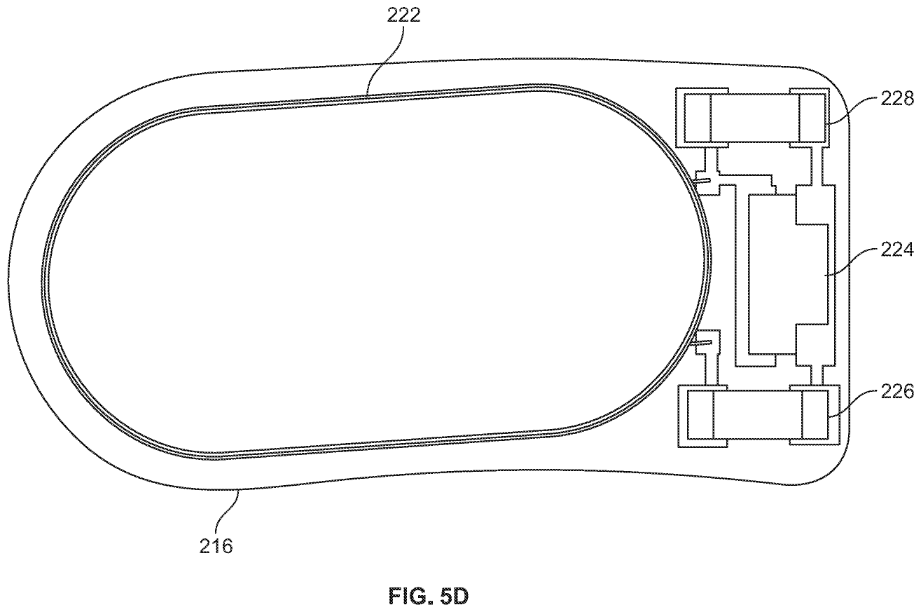

FIGS. 3A-5D depict another electromagnetic cage 200 according to the disclosure. EM cage 200 can include a body 202 with an open interior and open top and a corresponding lid 204 configured to attach to the body 202 to cover the open interior. The body 202 can include a hollow chamber 206 defined partially or completely through the body 202 and the lid 204 can include a correspondingly shaped aperture 208. Together, the hollow chamber 206 in the body 202 and the aperture 208 in the lid 204 can define a bone graft chamber 210 that can be configured to receive bone graft material to aid in bone fusion. A threaded inserter recess 212 can also be defined in body 202 to receive a distal end of an insertion tool used to insert the EM cage 100 into the body through the surgical access opening. In embodiments, the body 202 and/or lid 204 can have a textured outer bearing surface 214 to provide grip and traction against the bone to prevent accidental movement or withdrawal of the EM cage 200 and/or to promote bone growth into bearing surface 214 to better retain EM cage 200. In embodiments, cage 200 is sized and shaped for use as an Transforaminal Lumbar Interbody Fusion (TLIF) cage.

Open interior of body 202 can be configured to contain the electrical components for creating the electromagnetic field within and/or around the bone chamber 210. Breadboard or base 216 for the electrical components can be shaped to be retained by a raised wall 218 and an inner ring 220 defined in body 202. A metallic coil 222 can extend from breadboard for generating the electromagnetic field. Coil 222 can have a generally ovoid configuration defining an open interior matching the bone graft chamber and fitting around the inner ring 220 of body 202. In one embodiment, coil 22 can have a width of approximately 13.8 mm, a depth of approximately 10 mm and a height of approximately 7.5 mm. Breadboard 216 can further include can include a an ultrasonic transducer 224 that transfers power to the coil 222, a MCU (microcontroller) chip 226 that controls the electrical operations of the device including in embodiments wireless communications such as by Bluetooth, for example, and a power supply 228 that provides power to the transducer 224.

In embodiments, the ultrasonic power transducer 224 can be a piezo-electric energy circuit that will achieve up to 70% efficiency providing an average of 5-10 mw of power within a 1 cm² area. In embodiments, the power consumption of the power supply 228 and MCU 226 chips can be configured to minimize power consumption. In addition, the frequency that drives the coils can be kept below 100 khz to minimize energy consumption.

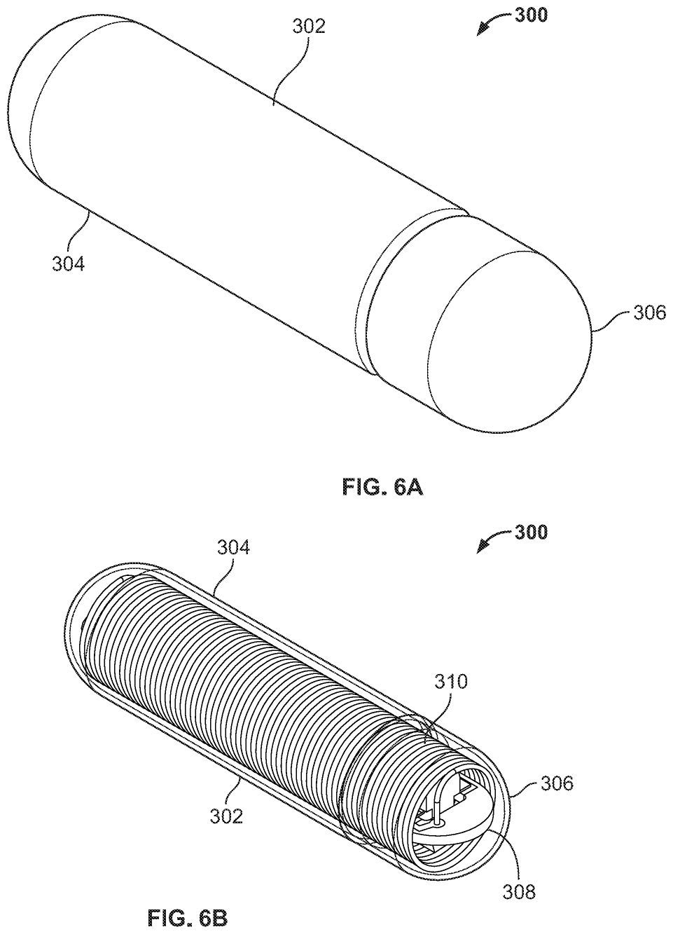

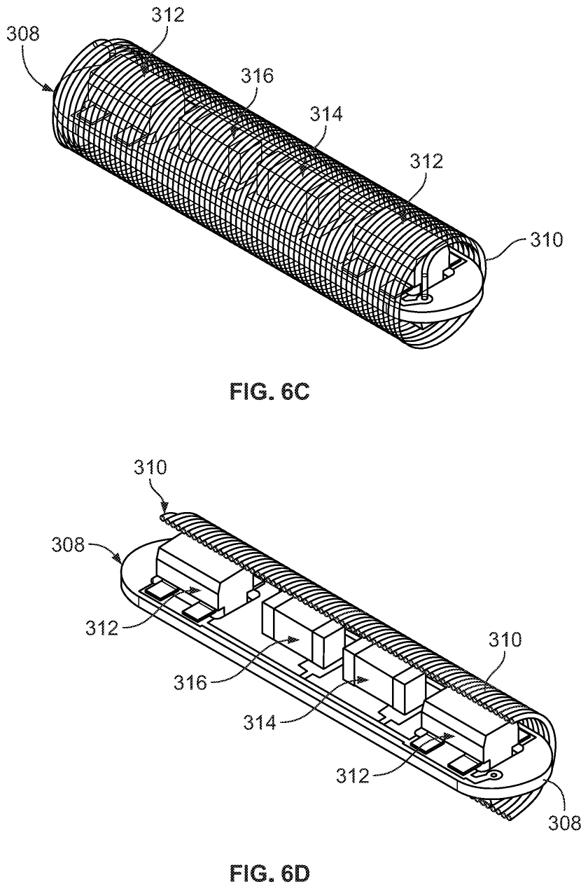

An electromagnetic insert 300 according to an embodiment of the disclosure is depicted in FIGS. 6A-7. In embodiments, EM insert 300 is generally bullet or pill shaped. EM insert 300 can include a protective cover 302 having a hollow interior for containing the electrical components that generate the electromagnetic field. Cover 302 can include a body 304 and a lid 306. Internal electrical components can include a base or breadboard 308 and a surrounding metallic coil 310. Breadboard 208 can further include a pair of transducers 312 that energize the coil 310 with power supplied by power supply 314. An MCU 316 can control operations of the device, including in some embodiments wireless communication via, for example, Bluetooth. In embodiments, coil 310 can have a length of approximately 10 mm and a coil diameter of between 2 mm and 5 mm. A 10 mm coil with 5 mm diameter will have N=20 turns.

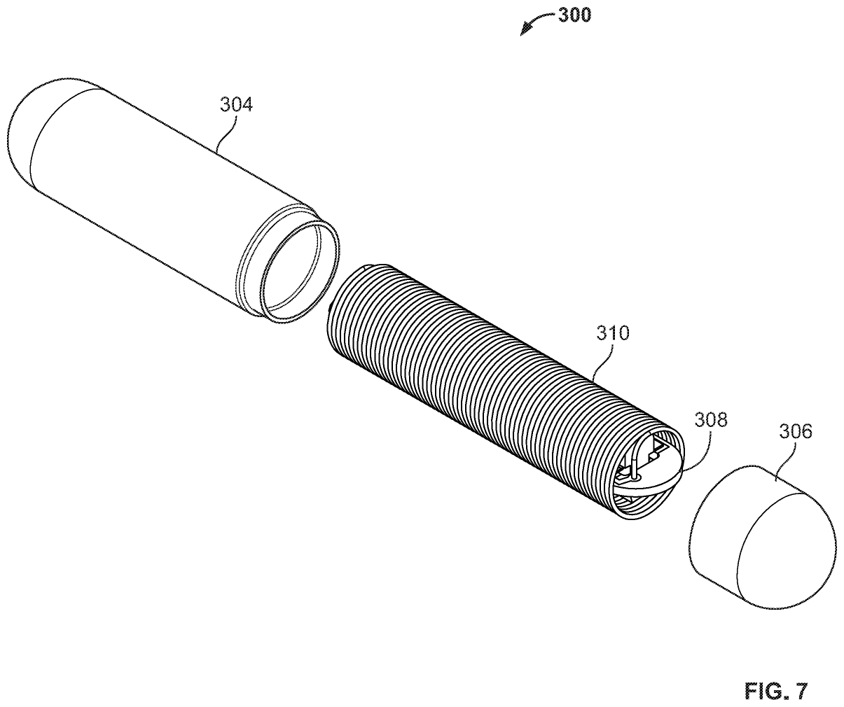

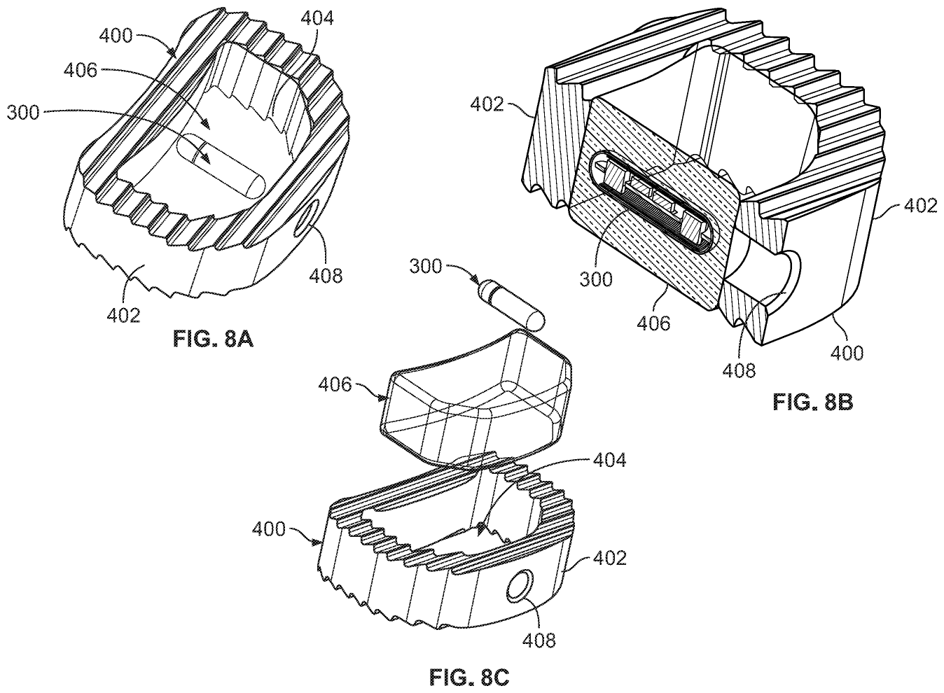

Referring now to FIGS. 8A-8C, in embodiments EM 300 insert is configured to be inserted into a bone matrix material within a bone chamber of an intervertebral cage. Intervertebral cage 400 can include a body 402 defining a hollow chamber 404. Upon implantation, the hollow chamber 404 can serve as a bone chamber configured to receive bone graft material 406 to aid in bone fusion. In embodiments, body 402 can further define an aperture 408 through which EM insert 300 is configured to be inserted in order to be embedded within the bone matrix 406. In one embodiment, body 402 has a width between 35 to 40 mm, a depth between 25 to 30 mm and a height between 7 to 12 mm.

In embodiments, the bone matrix 406 device can be, for example, either a porcelain matrix, or human cadaveric allograft or calcium carbonate matrix or even human cadaveric bone that would act as a scaffold to house the electrical circuitry. The device would be assembled by adding layers of the raw matrix layers that will also help induce bone growth. The bone matrix can take many shapes that could be implanted in a cage as a small square, cylindrical or tubular configuration. Also, in one configuration the device can be implanted through a trocar hole made in the pedicle reaching the center of the vertebra via a cannula.

Each of the above configurations, including ALIF cage 100, TLIF cage 200 and EM insert 300 inserted within cage 400 was tested for magnetic field generation and power requirements. The magnetic field generated by each device is dependent on the geometry of the coil. With respect to ALIF cage 100, it was found that the implanted coil geometry was similar to that of a solenoid and that the power required to generate tens of milliTesla was in the hinders of milliWatts range. For TLIF cage 200, it was found that the implanted coil generates sag toward the center due to its oblong shape and also generated tens of milliTesla with hundreds of milliWatts of power. With both cages, it was found that the coils generate large magnetic fields inside the coil and small fields outside the coil. As such, in some embodiments the cages are positioned such that the coil is positioned around the desired region for bone growth. It was also determined that preferably the coil height to coil diameter ratio be at least one. In some embodiments, the height to diameter ratio may be 2:1. It was further found that the magnetic field decays rapidly away from the EM insert and that while the implant could be driven with larger currents to increase the field distributed externally, such an approach would be highly inefficient.

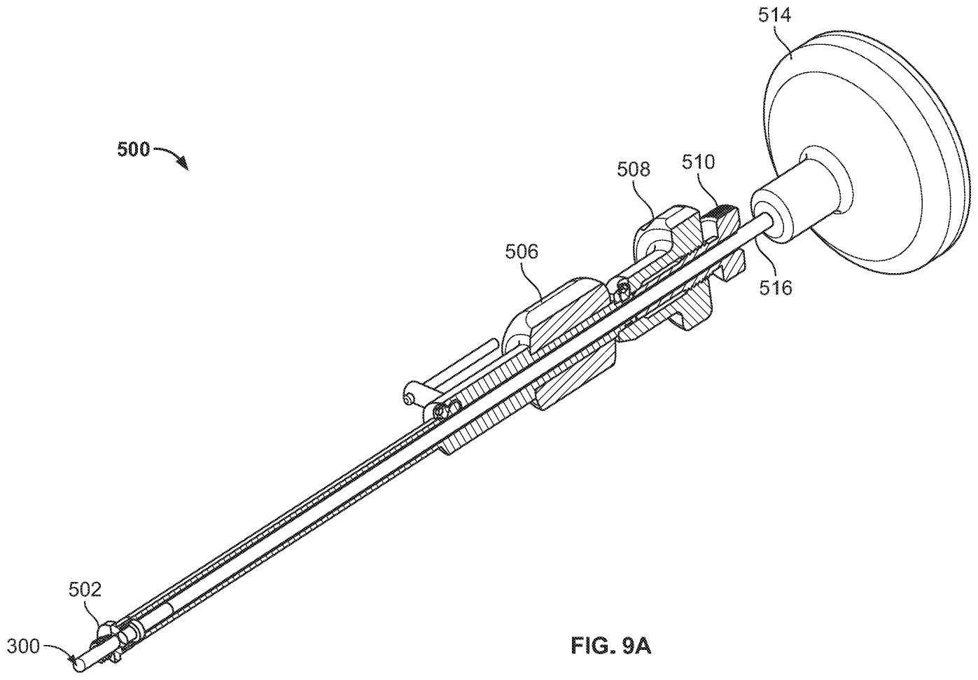

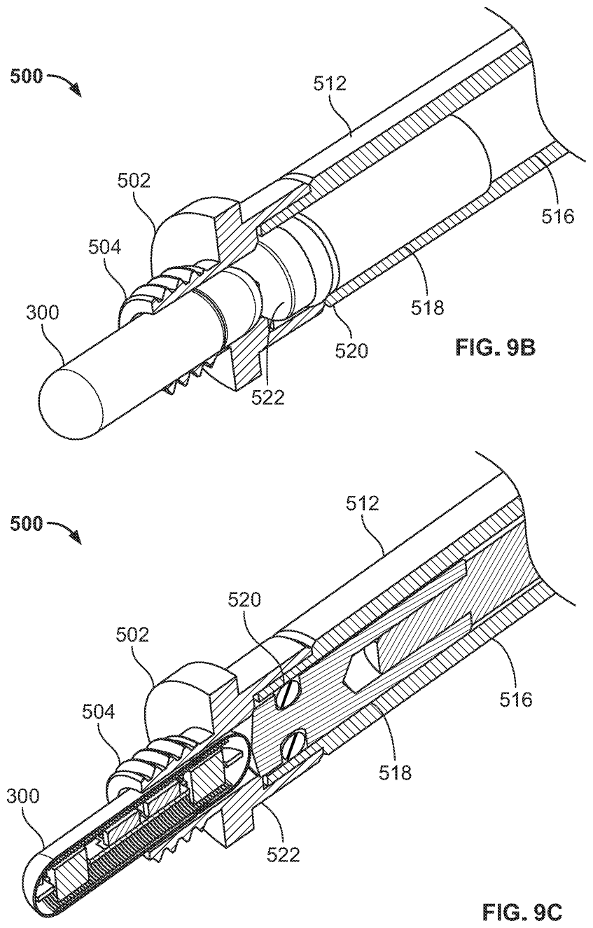

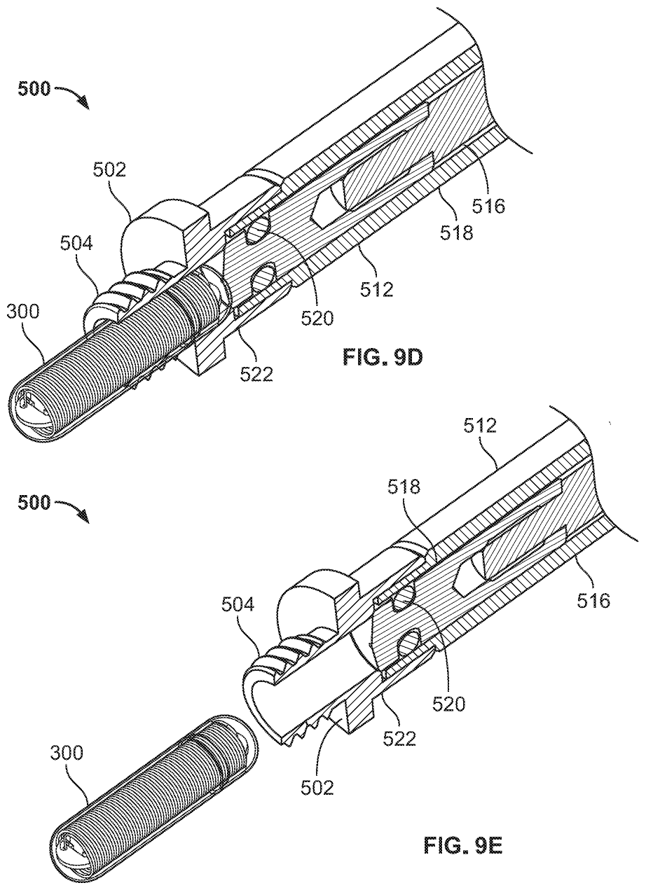

FIGS. 9A-9E depict an embodiment of an insertion device 500 for inserting an electromagnetic implant such as implant 300 according to an embodiment of the disclosure. Inserter 500 includes a distal tip 502 defining a channel configured to retain implant 400 therein. In an embodiment, channel is configured to retain implant via a friction fit. Tip 502 can include a threaded end 504 configured to be rotated to releasably attach distal tip 502 to an aperture in an electromagnetic cage such as aperture 408 in cage 400. In other embodiments, insertion device 500 can be used without an implant such as implant 300 and distal tip 502 can attached to an aperture of an EM cage such as aperture 110 of EM cage 100 or aperture 212 of EM cage 200 to insert an EM cage into a disc space. In embodiments, distal tip 502 can be rotated to attach threaded end 504 to an EM cage with one or more handle devices, which can include, depending on the specific configuration of the various inserter 500 components, handle 506, handle 508 or handle 510. In the depicted embodiment, handle 510 includes a shaft 512 that extends to distal tip 502 such that rotation of handle 510 causes distal tip 502 to rotate. Further details regarding such inserters and various components that can be included in and functions provided to such inserters can be found in U.S. patent application Ser. No. 16/292,565, which is hereby incorporated by reference in its entirety.

Inserter 500 can further include a plunger handle 514 connected to a plunger rod 516 that extends from the proximal end of inserter 500 through shaft 512 towards the distal end of the inserter 500. Plunger shaft 516 attaches to a plunger end 518 that can be held slidably held within shaft 512 with an o-ring 520. Plunger end 518 can further include a plunger tip 522 configured to abut a proximal end of the implant 300. After the threaded end 504 of tip 502 has been attached to aperture 408 of cage 400 and the cage is within the disc space, plunger handle 514 can be actuated to advance plunger shaft 516 distally. Plunger tip 522 will then drive the implant 300 out of the inserter 500 and into the opening 404 and/or bone matrix 406 within the cage 400. The threaded end 504 of inserter 500 can then be unscrewed from cage 400 and the inserter 500 removed.

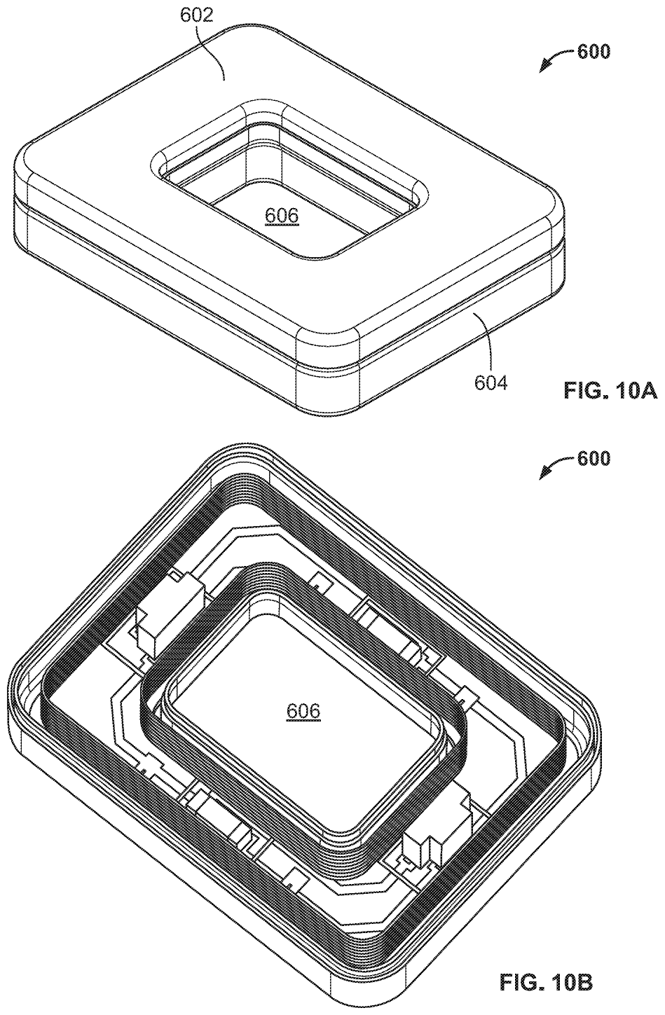

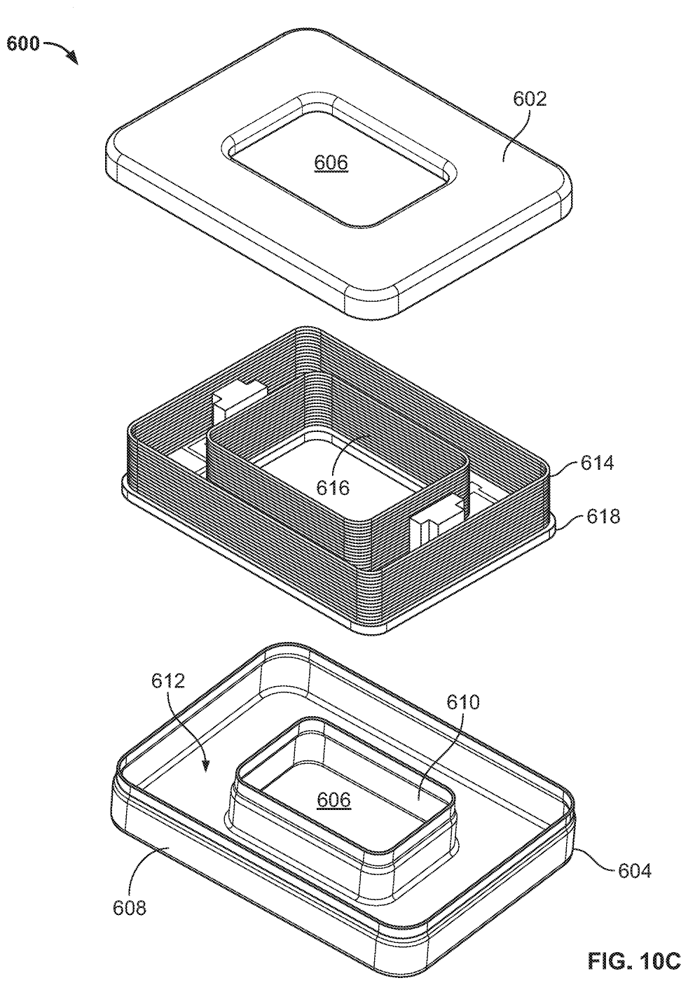

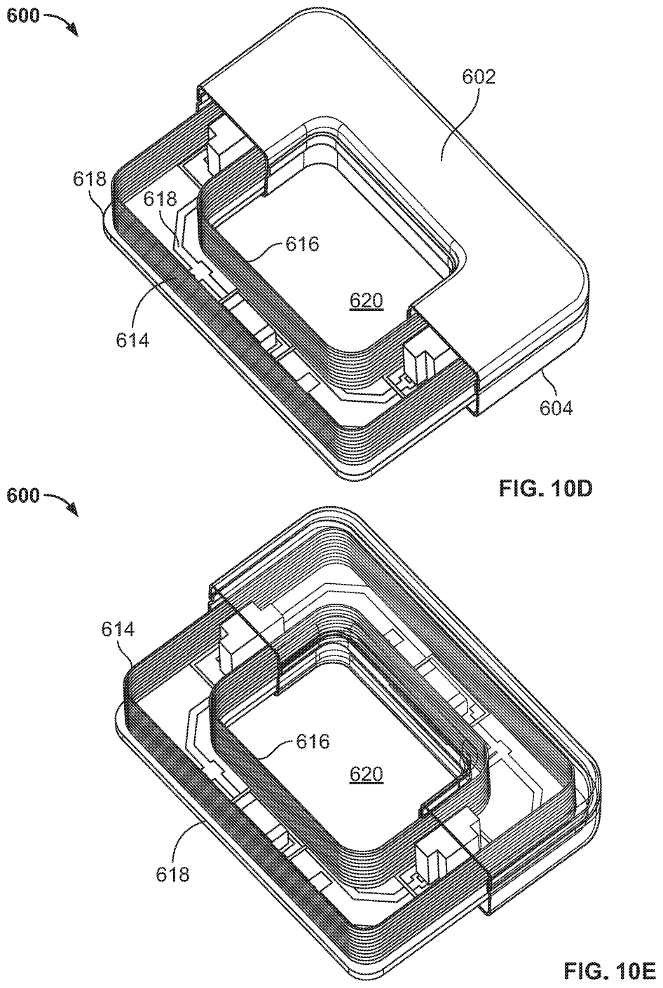

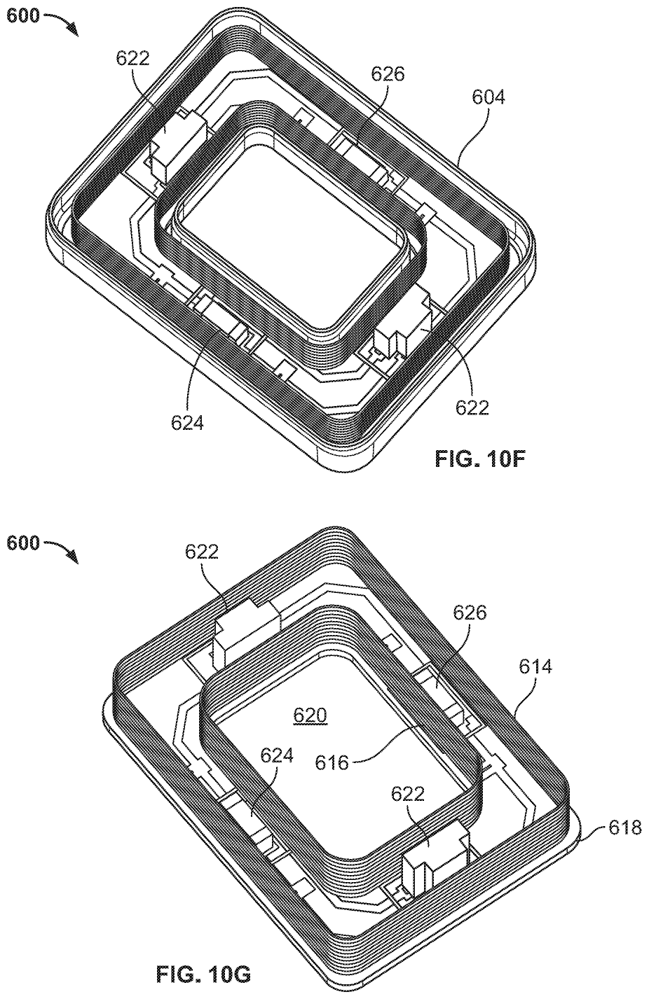

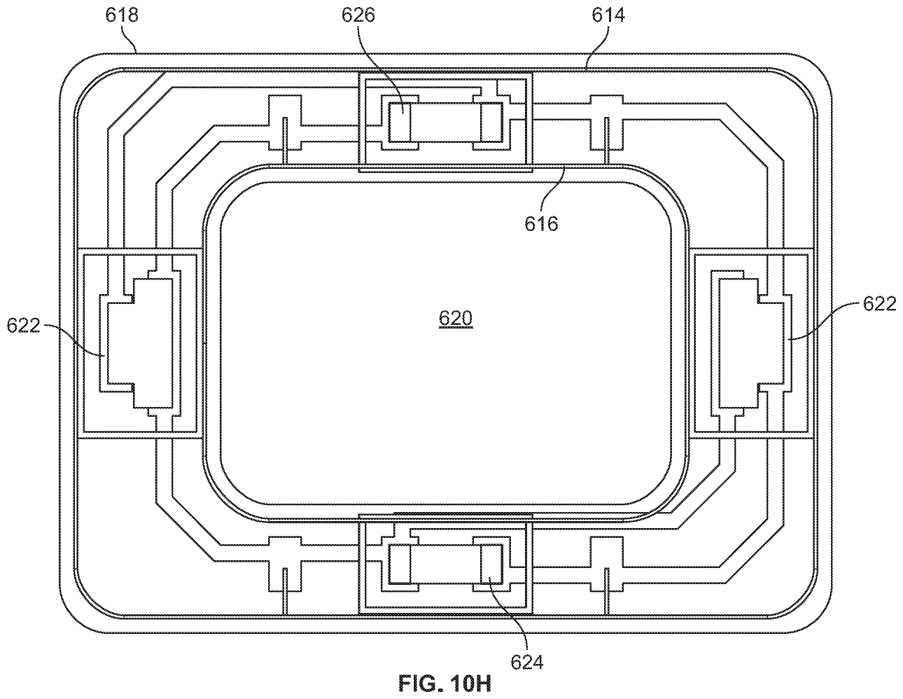

FIGS. 10A-10H depict an electromagnetic cage 600 according to another embodiment of the disclosure having a dual coil configuration. EM cage 600 can include an upper portion 602 and a lower portion 604. Each of upper portion 602 and lower portion 604 can define an inner chamber 606 having corresponding shapes. Lower portion 604 can define a concentric outer wall 608 and inner wall 610 that define a component chamber 612 therebetween configured to contain the electromagnetic components of the cage 600. Upper portion 602 can also include a corresponding chamber that provides room for a portion of the electromagnetic components within the upper 602 and lower 604 portions. Inner wall 610 further defines the inner chamber 606.

EM cage 600 can include both an outer metallic coil 614 and an inner metallic coil 616 mounted on a breadboard 618 sized to fit within component chamber 612. Breadboard 618 can include an inner chamber 620 matching inner chamber 606 and configured to be disposed around inner wall 610. Breadboard 618 can further can include a pair of ultrasonic transducers 622 that transfer power to the coils 614, 616, a power supply 624 that provides power to the transducers 622 and a MCU (microcontroller) chip 626 that can control device operations including in embodiments wireless communications such as by Bluetooth, for example. The dual coil configuration can extend a range of the electromagnetic field with respect to single coil embodiments. For example, whereas the electromagnetic field may in embodiments be primarily contained within the bone chamber of single coil embodiments, with dual coil configurations the electromagnetic field may further project beyond the exterior and outer perimeter of the cage. In embodiments, coils 614, 616 can be connected in parallel. In alternate embodiments, a similarly configured device can be provided with only a single coil. In one embodiment, the cage 600 measures 26×20×5.5 mm with inner chamber 606 measuring 8.6×12.6 mm.

Energy must be provided to power the coils of the devices disclosed herein, whether from a rechargeable internal power supply or via an external power source. Because the devices are implanted within the body, the energy for recharging or powering the coils must be provided wirelessly from outside the body. In various embodiments, power can be provided to the devices disclosed herein with either ultrasound or magnetic coils. Magnetic coils for providing power to the devices disclosed herein provide the advantages of not requiring a specific alignment with the implanted device with some geometries and can penetrate bone and air with minimal power losses in tissue. Magnetic coils also transfer energy efficiently if the coils are large enough. Ultrasound can be more sensitive to alignment and requires an acoustic interface for the transmitter. Power loss increases exponentially with the depth of the implant in soft tissue and there can be high energy loss at tissue-bone interfaces. Ultrasound can work well for small implanted devices (mm scale).

In embodiments, basic requirements for EM cages according to the disclosure include a minimum electromagnetic field strength of 10 Gauss (1 milliTesla) within the bone chamber. In various embodiments, the energy provided by a pulsed electromagnetic fields (PEMF) or by a static or constant magnetic field. PEMF can provide a magnetic field on the order of or less than 10 mT where as a static magnetic field can provide 50 mT or greater. PEMF has much lower energy consumption unless the static field is permanent or enhanced with ferromagnetic. In an embodiment, the energy is provided at 75 Hz. For PEMF, power can be provided at 250 mW or lower. In various embodiments, the preferred method of powering the implant is through electromagnetic induction.

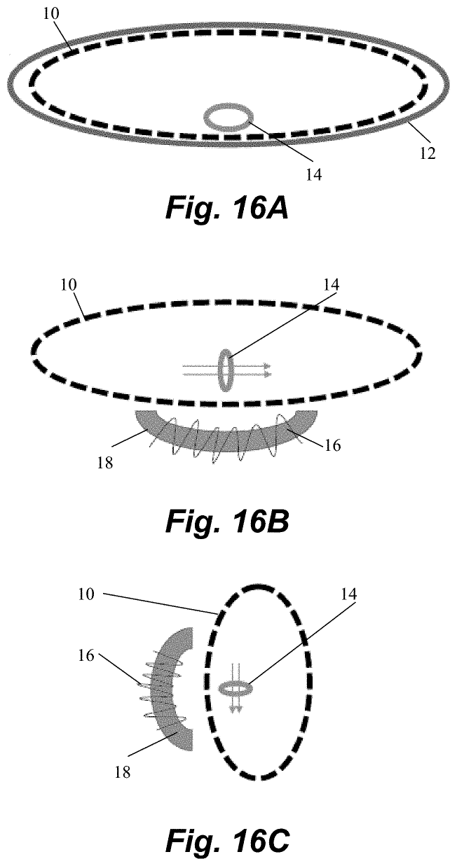

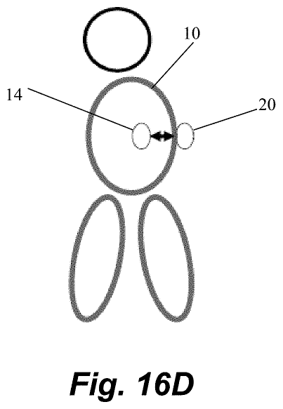

In an embodiment, power can be transferred to the implantable devices disclosed herein with a power belt 12 including electromagnetic coils within the belt as schematically depicted in FIG. 16A. The power belt would be worn all the way around the waist 10 of the user and power the implant via electromagnetic induction. The implanted coil 14 could lie in a horizontal position for receiving energy, which enables the device to be implanted with a low vertical profile within the body. The power belt 12 can have a height sufficient to make vertical alignment of the belt and the implant automatic such that the system is insensitive to vertical alignment. However, the large internal electromagnetic field generated by the belt may be less efficient for power delivery and will expose additional tissue to electromagnetic fields, potentially increasing the risk of undesired side effects. In one embodiment, the implanted coil of the device can include a core material that helps concentrate the magnetic field.

In another embodiment, a power belt worn around the waist 10 for transferring power to an implant can include a coil configured as an external winding 16 around a magnetic core 18 within the power belt to help direct the magnetic field. Although such a configuration as depicted in FIG. 16B would likely lead to increased efficiency, the internal coil 14 would need to be in a vertical orientation increasing the vertical footprint of the device within the body. In embodiments, the power belt could be worn around the entire waste with the magnetic core configured horizontally along the back of the user rather than extending around the entire belt. Alternatively, as depicted in FIG. 16C, the magnetic core 18 and external winding 16 could have a vertical orientation along the user's back. Such a configuration would provide increased efficiency due to the magnetic core while allowing the coil to be in a lower profile horizontal orientation in the body.

In another embodiment, power can be transferred to the devices disclosed herein with coaxially aligned coils. Referring to FIG. 16D, an individual coil 20 could be worn or otherwise temporarily held against the body 10 of the user in coaxial alignment with the internal coil 14. Such a configuration would be highly efficient using a resonant inductive configuration. However, the internal coil would have to have a greater vertical height that the above-described power belt approaches to aid in proper alignment and the configuration would require such proper coaxial alignment.

In other embodiments, ultrasonic energy can be used to provide power to the device. Use of ultrasonic energy is dependent on the implant geometry with respect to the distance between the ultrasonic transmitter and the device receiver as well as the presence of bone in the pathway from the ultrasonic transmitter to the device receiver. With greater distance between the transmitter and receiver and with presence of bone efficiency is lost.

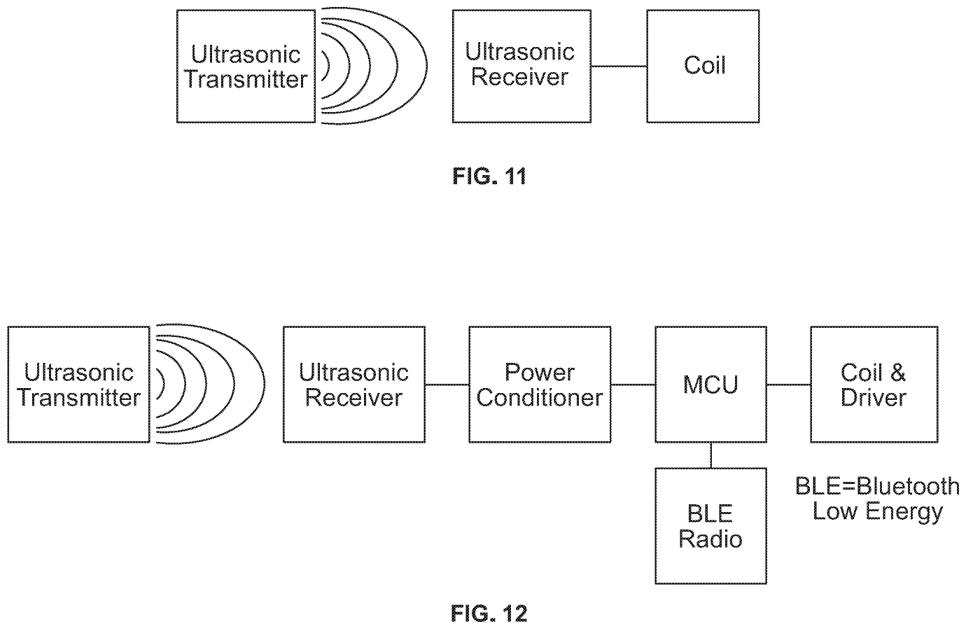

In one embodiment, the source for powering the device can include an ultrasonic transmitter and receiver. The coil current could be a pulsed, variable current having a duration of 1 to 1000 ms at an interval of 10 ms to 10 seconds. A schematic representation of such a basic device is depicted in FIG. 11. As can be seen in the figure, this basic device requires an ultrasonic transmitter to power the ultrasonic receiver, which is directly connected to and directly powers the coil, as there is no internal power source or control element.

FIG. 12 depicts a schematic representation of another embodiment consistent with the electromagnetic cages described above. In this embodiment, power transferred from the external ultrasonic transmitter to the ultrasonic receiver can be stored in the power conditioner to enable the device to be used in the absence of the transmitter and to be periodically recharged as needed. The power from the power conditioner is used to power the coil, the MCU and the Bluetooth radio or other communication device. In embodiments, the power conditioner must supply a stable DC voltage of at least 1.8V to the MCU. The average power consumption of the system is expected to be around 2 or 3 mw, with peaks of up to 10 mw. These will be in the range for the baseline requirements for the output of the transducer and power supply subsystem. In one embodiment, the strongest magnetic field for a given input level can be obtained using a coil of 500 turns, with a 4.7 of capacitor in parallel. Larger coils tend to produce stronger fields, but only if the coil is operated below the self-resonant frequency. The power consumption of the controller and MCU chips will be developed to minimize power consumption. In addition, the frequency that drives the coils will be kept below 100 khz to minimize energy consumption.

A similar configuration of the ultrasonic transmitter and ultrasonic receiver can be employed in the embodiments of both FIG. 11 and FIG. 12. In embodiments, the power received by the ultrasonic transceiver is proportional to the surface area of the transducer(s) employed. However, the transducers must be small enough to fit inside an appropriately sized cage. In an embodiment, the estimated available surface area for the transducers is 1 square centimeter. Thus, in embodiments, the cage will include a pair of transducers each about 0.5 square centimeters each. In embodiments, the optimum load impedance for maximum power transfer is 1000 ohms. In some embodiments, the ultrasonic power transducers that are embedded in the bone matrix implant or spinal cage can be a piezo-electric energy circuit that will achieve up to 70% efficiency providing an average of 5-10 mw of power within a 1 cm² area.

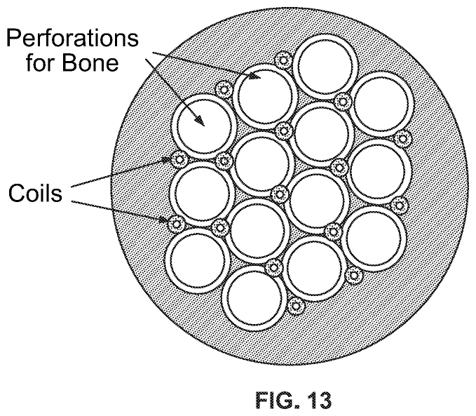

With initial reference to the EM cage embodiments described above, the coil configuration for EM cages can be implemented in various ways. In embodiments, device can include a single coil surrounding the internal bone chamber of EM cage. In some such embodiments, the strongest magnetic field has been shown to occur in the center of the bone chamber, with the field being about half the maximum strength at the outer perimeter of the chamber. FIG. 13 depicts a schematic representation of an alternative embodiment that employs a micro-coil array embedded in the bone chamber. Referring to FIG. 13, such an EM cage can include a plurality of micro-coils electrically connected with each other with perforations for bone growth and/or bone growth material therebetween. Such a configuration can provide a more even distribution of the magnetic field and more control over the total impedance and DC resistance in the system. In various embodiments, the micro-coils can be connected in parallel to lower impedance or in series to increase impedance.

In various embodiments, the coil(s) employed with EM cages according to the disclosure can be driven with either alternating current (AC) or direct current (DC). In embodiments, the passive device of FIG. 11 can be driven with AC, which increases the total impedance, as does a resonant capacitor that can be in parallel with the coil, requiring higher drive voltages ranging from about 1V to 10V and higher drive currents ranging from 10 mA to 100 mA. The coil size for such embodiments can range from about 80 to turns to 800 turns, with the number of turns depending on the drive current. For example, 80 turns @ 100 mA=800 turns @ 10 mA=8 Ampere-Turns

Embodiments such as those depicted in FIG. 12 can be driven with DC, which can limit the drive current to 1 mA to 2 mA and the drive voltage to a minimum of 1.8V. This reduced drive current requires a greater coil size. For example, 8000 turns @ 1 mA=8 Ampere-Turns. The power conditioner of such embodiments preferably has an efficiency of 80% or better and the minimum output voltage of 1.8 V. In embodiments, power conditioners providing additional voltage, such as, for example 2.5 V or 3.3V can enable greater functionality for the MCU and Radio. In various embodiments, power conditioner may need to be able to provide a peak current up to 10 mA with an average current of between about 3 mA to 5 mA. In embodiments, power conditioner can have a size of 3 mm by 5 mm.

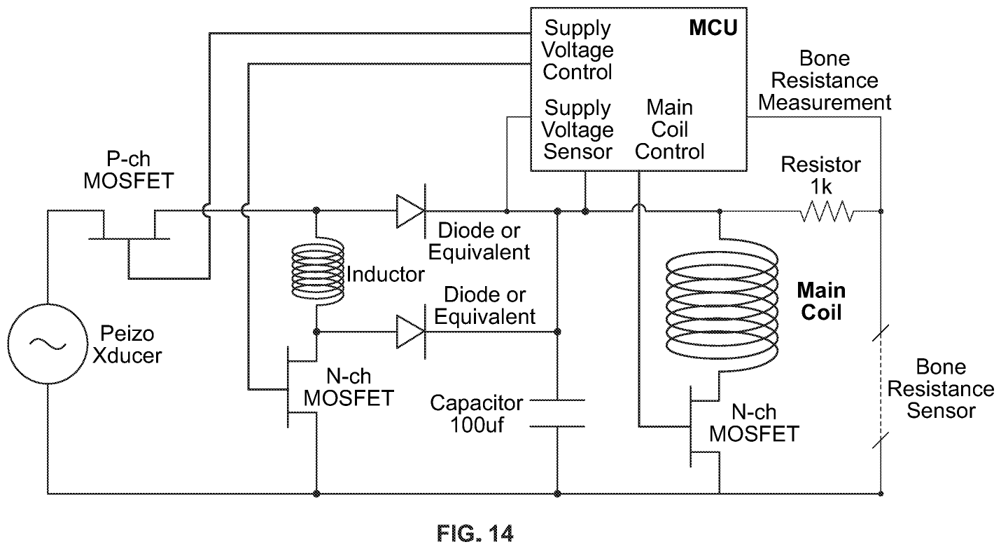

In some embodiments, EM cage can employ a piezoelectric energy harvesting circuit. One embodiment of such a circuit is depicted in FIG. 14. In the depicted embodiment, the power supply control and the switching waveforms for piezo harvesting are supplied by the MCU. In embodiments, a small battery may be employed to initiate the system. In an alternative embodiment, a hybrid approach could be employed with external coils.

In embodiments, the MCU and radio are preferably configured to utilize as low an average current consumption as possible and be small in size. In embodiments, MCU and radio can be approximately 3 mm×5 mm. MCU con be configured to have a sleep mode from which the device awakens based on communications with the radio and the ability of a programmer to communicate with MCU to power down unused subsystems. In various embodiments, MCU can operate at 1.8V, 2.5V or 3.3 V. In embodiments, maximum current consumption of MCU can be up to 10 mA with an average current of between about 1 mA to 3 mA. In one embodiment, radio utilizes Bluetooth Low Energy (BLE), which provides the advantages of very low power consumption, easy integration with a smartphone application that can be configured for controlling MCU of EM cage and compatibility with 1.8V devices.

The initial firmware and software for EM cages described herein can consist mainly of low level control routines, such as controlling the amplitude and frequency of the stimulating waveform to the electromagnetic coils in the Bone Matrix device or spinal cage. The amplitude of the waveform can be controlled by loading different preset points. There can be a menu of arrays allowing the physician programmer to select from the menu the most desirable amplitude and waveform characteristics that best suits the patient based on baseline human standards of weight, muscle mass, adipose distribution, etc. The firmware design can have a large impact on the total power consumption of the device. The firmware can minimize the average current consumption and limit the peak current to maximize available power for electromagnetic production. In addition, the internal components can provide feedback including impedance, temperature, internal disc space pressure readings, spinal angulation measurements, micro calcium analysis, temperature for the purpose of understanding spine biomechanics, infection risk, fusion and pseudarthrosis. This data can be an adjunct to spinal X-rays and MRIs. The feedback can be in real time via a computer application in a smartphone or computer device.

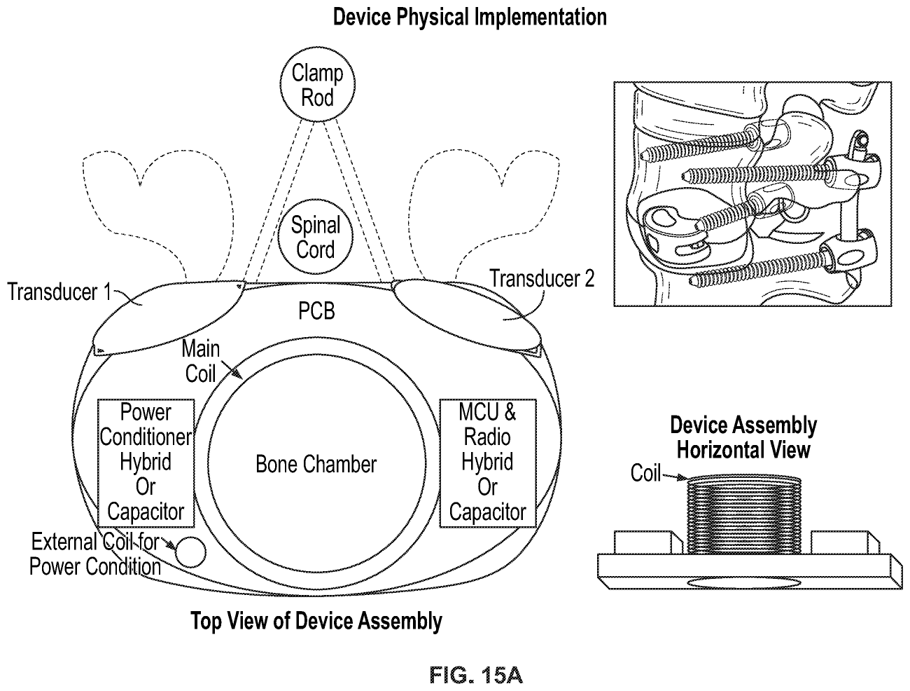

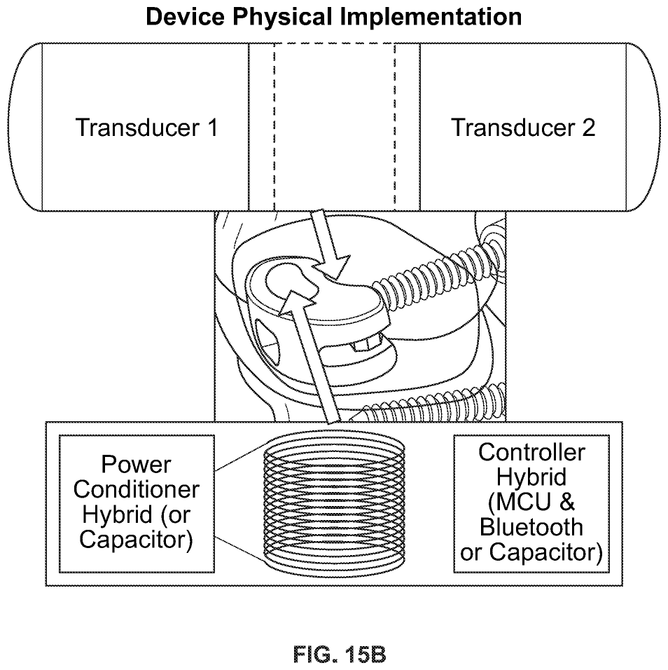

FIGS. 15A-15B schematically depict a physical implementation of an electromagnetic cage or implant according to the disclosure. Once access to the disc space is obtained, the EM cage is implanted between adjacent vertebrae. The bone chamber can then be filled with a bone matrix or other bone growth material. An external ultrasonic transmitter can be used to provide power to the ultrasonic receiver within the cage for powering the coil. In a passive embodiment such as that depicted in FIG. 11, the EM field for promoting bone growth is generated only when the external ultrasonic transmitter is present. In MCU controlled systems such as the system depicted in FIG. 12, the external ultrasonic transmitter can be used to provide power to be stored by the power conditioner in order to provide a continuous EM field within the bone chamber. Either way, the cage can remain within the patient following the procedure with the coil promoting bone growth within and around the bone chamber either continuously or periodically following the implantation procedure.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

1. An electromagnetic spinal cage configured to be implanted into an intervertebral disc space in a patient's body, comprising:
a device body having an interior chamber therein and having an open bone chamber defined therethrough;
a metallic coil disposed within the interior chamber and around the bone chamber; and
a power source configured to receive energy from an external transmitter and to supply power to the metallic coil to cause the metallic coil to generate a magnetic field within the bone chamber.

2. The electromagnetic spinal cage of claim 1, further comprising an external transmitter configured to supply power to the metallic coil.

3. The electromagnetic spinal cage of claim 2, wherein the external transmitter supplies power to the metallic coil through electromagnetic induction.

4. The electromagnetic spinal cage of claim 2, wherein the external transmitter supplies power to the metallic coil through ultrasonic energy.

5. The electromagnetic spinal cage of claim 1, wherein the device body comprises an upper portion and a lower portion releasably held together with one or more fasteners.

6. The electromagnetic spinal cage of claim 1, further comprising an inserter recess defined in the device body, the inserter recess configured to releasably secure the device body to an inserter configured to insert the device body into the intervertebral disc space.

7. The electromagnetic spinal cage of claim 1, further comprising a transducer disposed within the interior chamber of the device body, the transducer configured to receive power from the power supply and transfer the power to the metallic coil.

8. The electromagnetic spinal cage of claim 1, further comprising a controller disposed within the interior chamber of the device body.

9. The electromagnetic spinal cage of claim 8, wherein the controller is configured to wirelessly communicate with one or more external devices.

10. The electromagnetic spinal cage of claim 1, wherein the bone chamber and an interior of the metallic coil define a generally circular shape.

11. The electromagnetic spinal cage of claim 1, wherein the bone chamber and an interior of the metallic coil define a generally ovoid shape.

12. A system for spinal surgery, comprising:
an electromagnetic spinal cage configured to be implanted into an intervertebral disc space in a patient's body, the electromagnetic spinal cage including a device body having an interior chamber therein and having an open bone chamber defined therethrough and a metallic coil disposed within the interior chamber and around the bone chamber; and
an external power source configured to transfer energy to the electromagnetic spinal cage to power the metallic coil to cause the metallic coil to generate a magnetic field within the bone chamber.

13. The system of claim 12, wherein the external power source supplies power to the electromagnetic spinal cage through electromagnetic induction.

14. The system of claim 12, wherein the external power source supplies power to the electromagnetic spinal cage through ultrasonic energy.

15. The system of claim 12, wherein the device body of the electromagnetic spinal cage comprises an upper portion and a lower portion releasably held together with one or more fasteners.

16. The system of claim 12, further comprising an inserter configured to insert the electromagnetic spinal cage into the intervertebral disc space and an inserter recess defined in the electromagnetic spinal cage configured to releasably secure the electromagnetic spinal cage to the inserter.

17. The system of claim 12, further comprising a transducer disposed within the interior chamber of the electromagnetic spinal cage, the transducer configured to transfer the power to the metallic coil.

18. The system of claim 11, further comprising a controller disposed within the interior chamber of the electromagnetic spinal cage.

19. The system of claim 18, further comprising an external device configured to wirelessly communicate with the controller.

20. The system of claim 12, wherein the bone chamber and an interior of the metallic coil define a generally circular shape or a generally ovoid shape.
